(19) United States
(12) Patent Application Publication
(10) Pub. No.: US 2017/0262694 A1
(43) Pub. Date: Sep. 14, 2017

(54) Point-of-care Sample Analysis and Microscopy for Liquid Samples

(71) Applicant: The Board of Trustees of the Leland Stanford Junior University, Palo Alto, CA (US)

(72) Inventors: Audrey K. Bowden, Stanford, CA (US); Nicholas Dwork, Stanford, CA (US); Mehdi Javanmard, West Windsor, NJ (US); Saara Anwar Khan, Mountain View, CA (US); Kiran Rana Magar, Santa Clara, CA (US); Matthew Alexander Millet, Palo Alto, CA (US); Gennifer Tanabe Smith, Stanford, CA (US)

(21) Appl. No.: 15/456,240

(22) Filed: Mar. 10, 2017

Related U.S. Application Data
(60) Provisional application No. 62/307,048, filed on Mar. 11, 2016.

(51) Int. Cl. G06K 9/00; G06K 9/20; G06T 7/90; G06T 7/80; G01N 33/18; G01N 21/25; G01N 21/27; G01N 33/48; G01N 33/49; G01N 33/493; G02B 21/36; G06T 7/73

(52) U.S. Cl. CPC G06K 9/00134; G02B 21/365; G06K 9/209; G06K 9/00147; G06T 7/90; G06T 7/80; G06T 7/74; G01N 21/251; G01N 21/255; G01N 21/274; G01N 33/48; G01N 33/49; G01N 33/493; G01N 33/18; G06T 2207/10056; G06T 2207/10024; G06T 2207/30204; G01N 2021/1776

(57) ABSTRACT

Improved colorimetric analysis of liquid samples is provided. A sample holder is used that delivers predetermined volumes of sample individually to each of several colorimetric test patches at the same time with a sliding action. An opaque housing is employed to prevent ambient light from reaching the test patches when color images of the test patches are acquired. Preferably, a mobile electronic device including a camera is attached to the opaque housing to acquire the images. Optical microscopy can be performed in addition to the colorimetric analysis.

### CROSS REFERENCE TO RELATED APPLICATIONS

This application claims the benefit of U.S. provisional patent application 62/307,048 filed on Mar. 11, 2016, and hereby incorporated by reference in its entirety.

### FIELD OF THE INVENTION

This invention relates to colorimetric analysis of liquids.

### BACKGROUND

Colorimetric analysis relies on the change in color of an object (e.g., a test patch on a dipstick) to determine concentration of analytes in liquid solution. One application of this approach is urinalysis. Although the basic principle is simple, there have been sufficient complications in practice to prevent such urinalysis from being readily performed at home or where resources are limited. Thus far it has typically been necessary to use specialty lab facilities to obtain reliable results. Accordingly, it would be an advance in the art to provide improved colorimetric analysis of liquid samples.

### SUMMARY

A sample holder is used that delivers predetermined volumes of sample individually to each of several colorimetric test patches at the same time with a sliding action. An opaque housing is employed to prevent ambient light from reaching the test patches when color images of the test patches are acquired. Preferably, a mobile electronic device including a camera is attached to the opaque housing to acquire the images.

This configuration advantageously avoids multiple sources of user error in colorimetric analysis. Delivery of predetermined volumes of sample to each test patch avoids problems relating to insufficient sample on the test patch and cross contamination. Removal of ambient light via the opaque housing advantageously simplifies automatic color recognition. Markers can be used to automatically signal when the sample is first delivered to the test patches. The system can automatically ensure that the color comparisons for each test patch are done at the correct time after delivery of the sample. This is an especially difficult task to do manually, since several patches may need to be read at the same time or at nearly the same time. In some embodiments, optical microscopy is performed in addition to this colorimetric analysis.

### BRIEF DESCRIPTION OF THE DRAWINGS

FIGS. 1A-C show a manifold according to an embodiment of the invention.

FIGS. 2A-C show a chassis corresponding to the manifold of FIGS. 1A-C.

FIG. 3A-C show filling the manifold of FIGS. 1A-C with a sample.

FIG. 4A-D show operating the manifold of FIGS. 1A-C to deliver controlled amounts of sample to colorimetric test patches.

FIG. 5 shows an embodiment of the invention.

FIG. 6 shows interlocking pieces for the opaque housing of FIG. 5.

FIGS. 7A-D show operation of optical markers to show when the sample is delivered to the test patches.

FIG. 8 shows experimental results of delivered liquid volume.

FIG. 9 shows automatic recognition of the optical marker signal that sample has been delivered to the test patches.

FIG. 10 shows timing results for several analytes in urine samples.

FIGS. 11A-B show a sample holder including a microscopy well.

FIGS. 12A-F show examples of particulates that may be imaged and (optionally) automatically recognized via microscopy combined with dipstick analysis.

### DETAILED DESCRIPTION

Section A describes general principles relating to embodiments of the invention. Section B is a detailed example of this approach as applied to urinalysis.

### A) General Principles

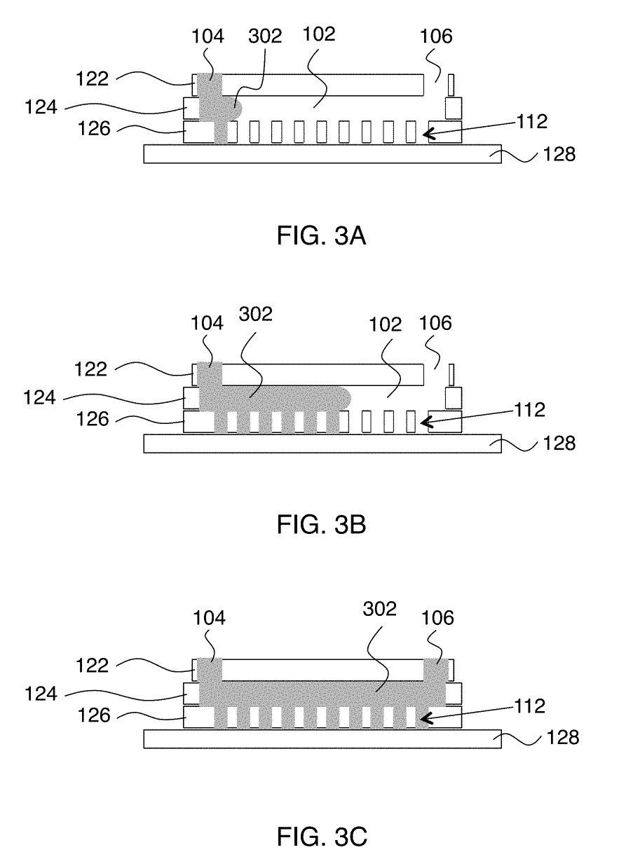

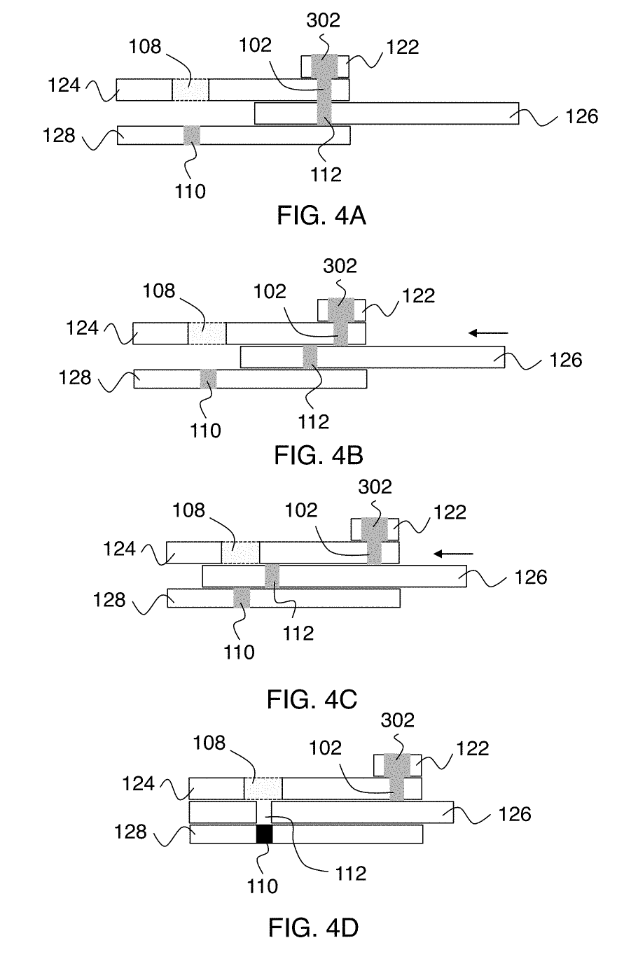

An exemplary embodiment of the invention is the apparatus for analysis of a liquid sample. The apparatus includes a sample holder configured to accept an analysis dipstick having two or more colorimetric test patches. The sample holder also includes a slidable mechanism having a first configuration (e.g., as in the example of FIG. 3A) where a liquid sample can be provided to a sample chamber in the sample holder and having a second configuration (e.g., as in the example of FIG. 4D) where a predetermined volume of the liquid sample is individually delivered from the sample chamber to each of the colorimetric test patches of the analysis dipstick. The apparatus also includes an opaque housing disposed to prevent ambient light from reaching the analysis dipstick when one or more color images of the analysis dipstick are acquired.

Preferably the apparatus further includes a camera configured to provide the one or more color images of the analysis dipstick. The camera can be included on a mobile electronic device. Alternatively, the camera can be integrated with the opaque housing. The color images are preferably acquired as a succession of video frames.

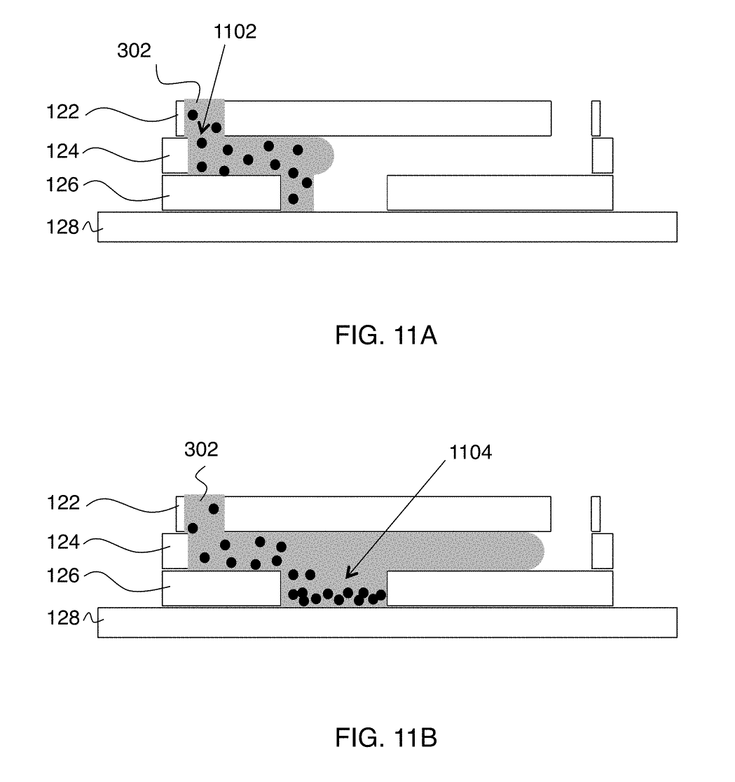

In some embodiments, optical microscopy of the liquid sample is provided in addition to dipstick analysis as described in detail below. FIGS. 11A-B show an exemplary sample holder for such embodiments. Here 122, 124, 126, 128 are layers as described below, and 1104 is a microscopy well in layer 126 configured to accumulate cells etc. 1102 from sample 302 for microscopic imaging. Microscopy well 1104 can be configured to act as a filter trench to accumulate cells and other particulates that may be present in liquid samples. Preferably microscopy well 1104 is upstream of the wells that are used for dipstick analysis (which are not shown on these figures).

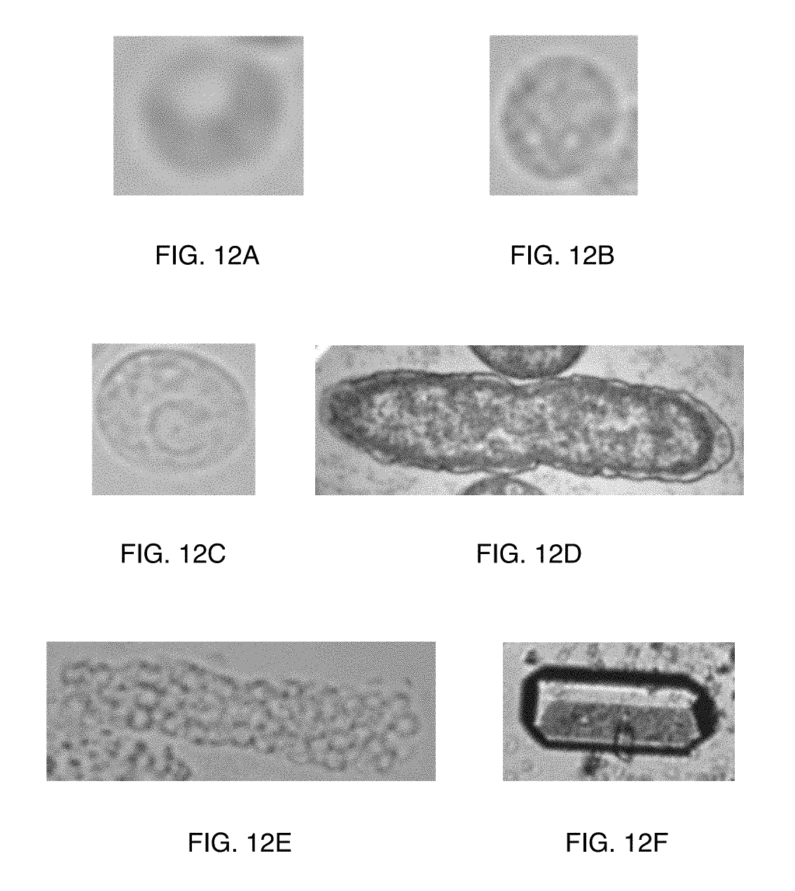

FIGS. 12A-F show some particulates that can occur in urine samples. FIG. 12A is an image of a red blood cell (typically 7-21 μm size) which confirms or may suggest rhabdomyolysis, intravascular hemolysis, hematuria, glomerular disease. FIG. 12B is an image of a white blood cell (typically 6-8 μm size) which confirms or may suggest pyuria (UTI), glomerulonephritis, bladder or pelvic inflammation. FIG. 12C is an image of an epithelial cell (typically 30-60 μm size) which confirms or may suggest acute tubular necrosis, exposure to nephrotoxic agents. FIG. 12D is an image of a bacterium (typically 2-6 μm length) which confirms or may suggest a urinary tract infection (UTI), prostatitis. FIG. 12E is an image of a cast which can signify various conditions according to the nature of the cast. FIG. 12F is an image of a crystal (typically 8-10 μm size) which confirms or may suggest kidney stones or liver disease.

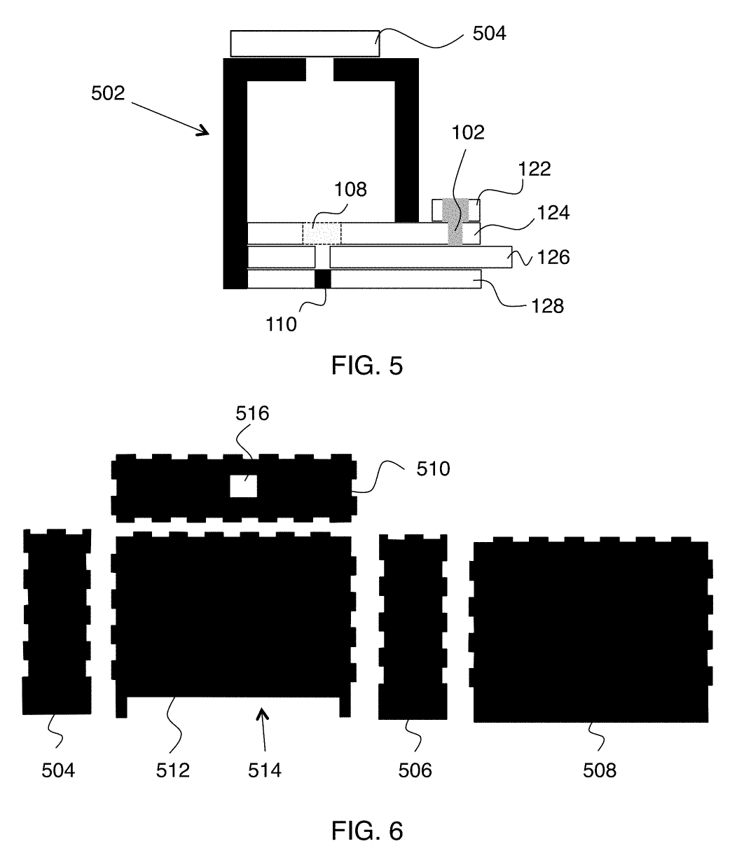

Embodiments of the invention including an optical microscopy feature preferably also include a processor configured to 1) provide automatic recognition of features of the magnified optical image and 2) provide results from the automatic recognition of features to a user. With reference to FIG. 5 below, 504 on that figure can be a combined processor and camera, such as an appropriately configured smart phone or other mobile device for performing these functions. Lenses and/or additional lighting can be provided for this microscopy, and such additions would be within the skill of ordinary art workers.

Although the detailed example given below relates to urinalysis, the invention is not limited to only the urinalysis application. This approach is applicable for any liquid sample to be analyzed via colorimetric test patches, including but not limited to: human urine samples, animal urine samples human blood samples, animal blood samples and water samples. Analysis of water samples includes but is not limited to: analysis of pool water and analysis of well water.

Embodiments of the invention can further include a processor configured to 1) provide a comparison of the one or more color images of the analysis dipstick to one or more reference images and 2) provide results from the comparison to a user. With reference to FIG. 5 below, 504 on that figure can be a combined processor and camera, such as an appropriately configured smart phone or other mobile device for performing these functions. The processor can be further configured to perform a color calibration. This color calibration can be a one-time calibration.

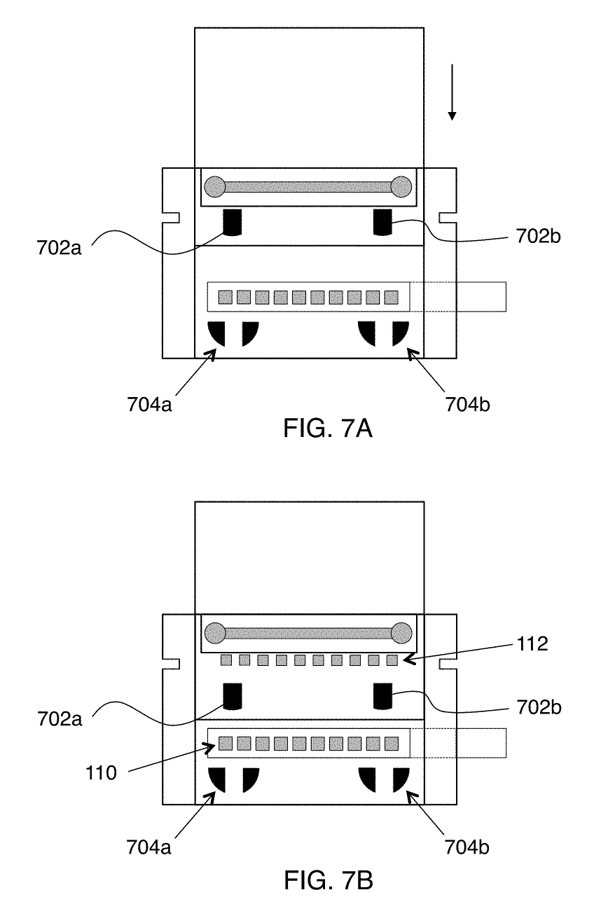

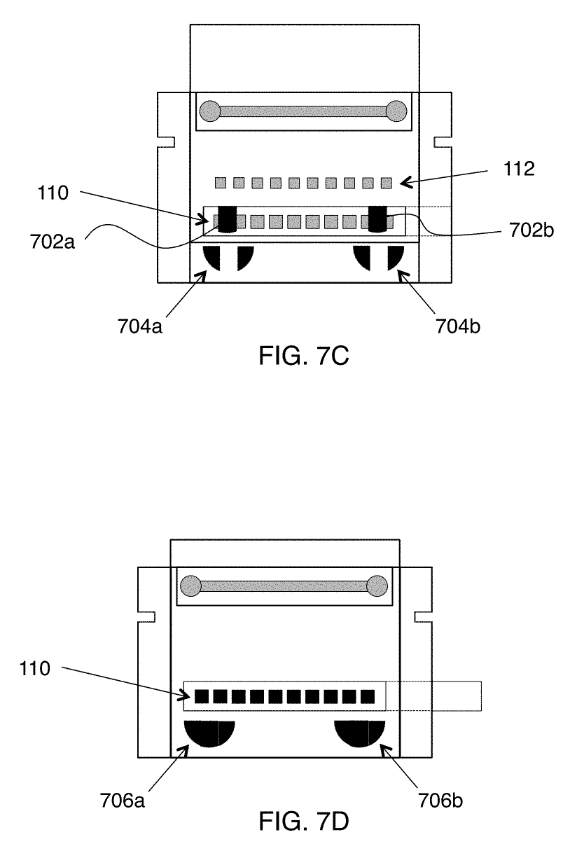

The sample holder can further include one or more optically recognizable markers having a first shape (e.g., the split markers 702a,b, 704a,b on FIG. 7A) when the sample holder is in its first configuration and having a second shape (e.g., 706a,b on FIG. 7D) when the sample holder is in its second configuration. Such embodiments can further comprise a processor configured to acquire the one or more color images of the analysis dipstick at one or more predetermined times after the one or more optically recognizable markers earliest have their second shape. In other words, the markers 706a,b on FIG. 7D earliest have their second shape right when the sample is delivered to the test patches, so that moment is the appropriate timing reference. With reference to FIG. 5 below, 504 on that figure can be a combined processor and camera, such as an appropriately configured smart phone or other mobile device for performing these functions.

The opaque housing can be configured as a plurality of planar members comprising plastic and having interlocking cutouts on their edges for assembly to form the housing. Here plastic is used as a generic term for a polymer material. Acrylic is an example of a preferred material for the opaque housing. Other suitable polymers are polymers that can be extruded or used in 3D printing technologies.

The sample holder can be configured as a stack of two or more planar members comprising plastic, and wherein the first and second configurations are defined by sliding one of the planar members relative to the other planar members. Here also plastic is used as a generic term for a polymer material. Acrylic is an example of a preferred material for the planar members. Other suitable polymers are polymers that can be extruded or used in 3D printing technologies.

The analysis dipstick can be a commercially available analysis dipstick, and the sample holder can be configured to mechanically mate with the commercially available analysis dipstick. As described in detail below, this mating may involve inserting test patches from the dipstick into receptacles of the sample holder.

### B) Experimental Demonstration

### B1) Introduction

Dipsticks for urinalysis are a convenient diagnostic tool: they are non-invasive, extremely portable, and very cheap to manufacture. Unfortunately, the accuracy of the results is highly dependent on 1) proper sample preparation, 2) correct interpretation of a gradient color scale, and 3) precise readout timing. Proper sample preparation includes carefully wiping the edge of the dipstick immediately after immersing it in urine to ensure application of the correct volume on each pad and to prevent the cross-contamination between adjacent pads that is common in the standard dip-and-wipe method. Interpretation of a gradient color scale often requires the user to differentiate between various shades of the same color, a difficult task for many people that can be exacerbated in certain lighting conditions. Additionally, the tests are inherently unreadable to users who are color-blind. Lastly, each pad of the dipstick must be read at a specific time to ensure accuracy, as the results vary over time. Several of the pads may have the same readout time, requiring the user to interpret several color results simultaneously. Furthermore, many of the other readout times are close together (within 10-30 s), so the user must be able to quickly determine the results to maintain precise readout times.

The stringent requirements of dipstick urinalysis thus makes the accuracy of at-home tests suspect. The problem, however, is not the test itself: dipstick urinalysis is a procedure commonly used in primary care settings that can simply and effectively screen for various diseases and conditions. Unfortunately, due to high user error, many clinicians will only utilize semi-quantitative dipstick results obtained by a trained technician, which typically requires sending the urine samples to a large, off-site testing facility. Such lab results may require a one- to three-day wait-time, even though the tests themselves can be run in a matter of minutes. A device that could be used at home and be trusted to produce accurate results would greatly reduce the burden on both the clinics and the general population: clinics would be able to dedicate their time and resources to higher risk patients (the frequency of negative test results is a significant burden on the healthcare system), and patients would be able to perform the tests in the comfort of their own homes, thus eliminating travel time and the need to schedule multiple appointments. Furthermore, tests could be run more often and results could be more easily tracked over time. Enabling high-fidelity, at-home urinalysis would be particularly useful because the first urinary discharge of the day is the ideal sample for certain tests (e.g., pH and nitrite testing for urinary tract infections). Moreover, robust and portable urinalysis tests would be extremely useful in low-resource settings that lack access to large testing facilities.

The use of mobile computing devices (e.g., cellular phones) has recently emerged as a novel strategy to overcome many of the hurdles that plague both existing dipstick tests and other strip-based tests. The accompanying software applications aim to standardize the color interpretation and timing of the dipstick results by guiding the user through the correct process and then capturing an image or video of the dipstick to perform automated color readings. Unfortunately, this strategy is still highly prone to user error, as the apps can only suggest the proper procedure but not actually control it. Several microfluidic-based urinalysis tests, take an altogether different approach and fabricate a proprietary platform to replace currently available dipsticks. However, broad and rapid adoption of these approaches may be hindered by the novelty of the testing platform and chemistry. Additionally, such platforms tend to still be susceptible to cross-contamination and timing errors.

In this work, we present a device and strategy for urinalysis that enables control over all aspects of the dipstick testing procedure. Importantly, unlike other microfluidic-based urinalysis systems, the newly developed device integrates with commercial, off-the-shelf dipsticks; the use of a well established chemistry platform makes it immediately suitable for at-home and low-resource settings. To facilitate proper sample preparation, we developed a microfluidic manifold capable of accurately depositing microliter volumes to each individual pad on the urinalysis dipstick. The isolation of the delivery mechanism prevents cross-contamination between adjacent pads. During operation, the manifold, including the dipstick, is housed in an opaque box to eliminate effects from changing lighting conditions. Finally, we developed offline software that utilizes a video captured by a mobile computing device to interpret the resulting colors of the dipstick pads and ensure accurate timing of the readout.

### B2) Experimental Design

### B2.1) Choice of Materials

For all urinalysis testing, we used Mission Urinalysis Reagent Strips (Acon) with pads for 10 analytes. Standard urine samples (Bio-Rad) were used for all testing; the urine samples are provided in two levels (level 1 and level 2), with information provided by the manufacturer regarding the ranges of analytes present in each sample. We chose to use these samples because they are readily available and are routinely used for calibrating automated dipstick readers used in clinics. In general, the analyte levels are lower in the level 1 sample compared to level 2. It should be noted that the ranges reported by the manufacturer encompass several possible candidate solutions; that is, the exact amount of analyte is not specified by the manufacturer, but rather a range of possible analyte concentrations is given that could be mapped to one of several possible pads (i.e., candidate solutions). We utilized a mobile phone (iPhone 4S) to capture all videos to mimic conditions likely available to many at-home users. Videos were analyzed with Matlab® software v R2013a (Mathworks), but could be integrated into a phone app in the future. We lasercut acrylic (Universal Laser System VLS 4.60 laser cutter) for all components of the device to reduce the cost of manufacturing.

### B2.2 Choice of Deposit Volume

To determine the optimal volume for accurate measurements, we deposited various volumes—5, 10, 15, and 20 μL—onto each dipstick pad using a micropipette, and the results were compared to the standard dip-and-wipe method. We used level 2 standard urine for all tests and analyzed all dipsticks using a color-matching technique described below. We validated the results by ensuring they were within the ranges provided by the manufacturer and by performing independent analyses by eye.

Median values and standard deviations over seven trials for each analyte and tested volume are given in Table 1. Note that the values are reported as the index value of the candidate solution for each analyte and not the candidate solution itself. Median values that differ from the medians obtained by the standard dip-and-wipe method are denoted by *, and the number of test pads whose median value differs from the standard is given in the bottom row. The data indicate that the volume sampled can impact the dipstick results; in particular, for the brand of dipstick tested, 15 and 20 μL result in only a single analyte pad whose value differs from the standard dip-and-wipe method. We chose 15 μL as the optimal volume, as overflow of the pads was often seen with 20 μL, leaving the dipstick susceptible to cross-contamination. It should be noted that while 15 μL is the optimal volume based on the closest match with the dip-and-wipe method for the brand of dipstick tested, different brands may have different optimal volumes based on the size of their test pads.

TABLE 1
Median values and standard deviations for all
analytes using various deposited volumes.

| | dip/wipe | 5 μL | 10 μL | 15 μL | 20 μL |
|---|---|---|---|---|---|
| LEU | 4 ± 0 | 3* ± 0 | | | |
| NIT | 2 ± 0 | 2 ± 0 | 2 ± 0 | 2 ± 0 | 2 ± 0 |
| URO | 4 ± 0.53 | 3* ± 0 | 4 ± 0 | 4 ± 0.49 | 4 ± 0.82 |
| PRO | 2 ± 0.53 | 3* ± 0.49 | 2 ± 0 | 2 ± 0 | 2 ± 0.53 |
| pH | 4 ± 0 | 3* ± 0 | 3* ± 0.38 | 4 ± 0.38 | 4 ± 0.53 |
| BLO | 5 ± 0 | 3* ± 0 | 4* ± 0 | 5 ± 0 | 5 ± 0 |
| SG | 3 ± 0.53 | 2* ± 0.53 | 1* ± 0.49 | 2* ± 0.38 | 2* ± 0.49 |
| KET | 4 ± 0.49 | 4 ± 0.38 | 4 ± 0.49 | 4 ± 0.49 | 4 ± 0.49 |
| GLU | 3 ± 0.58 | 1* ± 0 | 2* ± 0.58 | 3 ± 0.49 | 3 ± 0 |
| dif- | N/A | 7 | 5 | 1 | 1 |
| fer- | | | | | |
| ences | | | | | |
| vs. | | | | | |
| dip/ | | | | | |
| wipe | | | | | |

A * indicates median values that differ from the median obtained with the dip-and-wipe method, the industry standard. The total number of analyte pads whose medians differ from the dip- and-wipe method are reported in the bottom row.
Note results are cumulative for all dipsticks used.
LEU: leukocytes, NIT: nitrite, URO: urobilinogen, PRO: protein, BLO: blood, SG: specific gravity, KET: ketones, GLU: glucose.

When analyzing the Mission Urinalysis strips, the bilirubin (BIL) pad provided consistently erroneous results. We noted by eye that the observed pad colors (for various levels of BIL) and the colors in the reference chart did not match well. This observation was confirmed when we analyzed various urine samples using the color-matching technique described below. In fact, the BIL results always had significantly higher standard deviations than all the other pads, even when using the standard dip-and-wipe method (standard deviation=1.07). Due to this inconsistency in results, we do not report the results of the BIL pads in Table 1 or in subsequent results. This observation did not impact our choice of volume.

### B2.3) Device Design and Working Principle

We designed an all-inclusive urinalysis device including the following components: 1) a manifold that is capable of delivering specific volumes of the sample to each individual pad of the dipstick; 2) an acrylic box to house the manifold and shield it from external lighting; 3) a timing mechanism that interfaces with the manifold to identify the exact time at which a urine sample is deposited onto the dipstick pads; 4) software to analyze videos of the urinalysis procedure (timing mechanism and color change of dipstick pads) captured by a mobile computing device. Each component is discussed in more detail in the following sections.

### B2.3a) Dipstick Manifold for Volume Control

The manifold was inspired by the SlipChip, a device designed to transfer nanoliter volumes across a surface to designated areas (e.g., as described in U.S. Pat. No. 9,415,392, hereby incorporated by reference in its entirety). The original SlipChip is a two-layer device designed to perform multiplexed reactions without pumps or valves (e.g., recombinase polymerase amplification, polymerase chain reaction, and immunoassays); fabrication is fairly involved and it uses glass plates. The manifold of this work has several substantial differences relative to the SlipChip: 1) we use a four-layer (vs. two-layer) design; 2) it is fabricated using an all-acrylic framework (vs. glass); 3) it is optimized to transfer microliter (vs. nanoliter) volumes; 4) it integrates with a commercial dipstick. While our manifold works similarly to the SlipChip in that it is capable of transferring precise volumes across a surface using a slipping process, the differences in its design give it several significant advantages for application to urinalysis.

First, the four-layer design allows for easy integration with off-the-shelf dipsticks and simpler sample loading. Second, the choice of acrylic as a base material makes it more robust, cheaper, safer, and easier to produce. For instance, to create the layers (described below) out of acrylic cost approximately $0.85, whereas a similar design in glass would cost approximately $15. Note that these costs can be reduced if the devices are produced in bulk, and costs for our device of ˜$0.50 are conceivable. The high cost of a glass implementation is due to the inability to use standard glass slides, which are too small to hold the dipsticks. Additionally, acrylic can be fabricated using a laser cutter rather than a more costly lithography process. In contrast with photolithography suites, laser cutters are becoming ubiquitous due to their relatively low cost, speed, and ease of use. The lower cost and simpler fabrication make the manifold of this work more appropriate for primary care applications. Moreover, laser cutting enabled us to fabricate wells of arbitrary shape and provides versatility for use with multiple dipstick brands. Glass can also easily break if dropped on the ground and represents a potential safety hazard.

Third, the capability to handle microliters is necessary to handle the volumes needed for dipstick analysis. Fourth, the integration with existing dipsticks allows it to take advantage of chemistries that have been well established and used for more than 50 years.

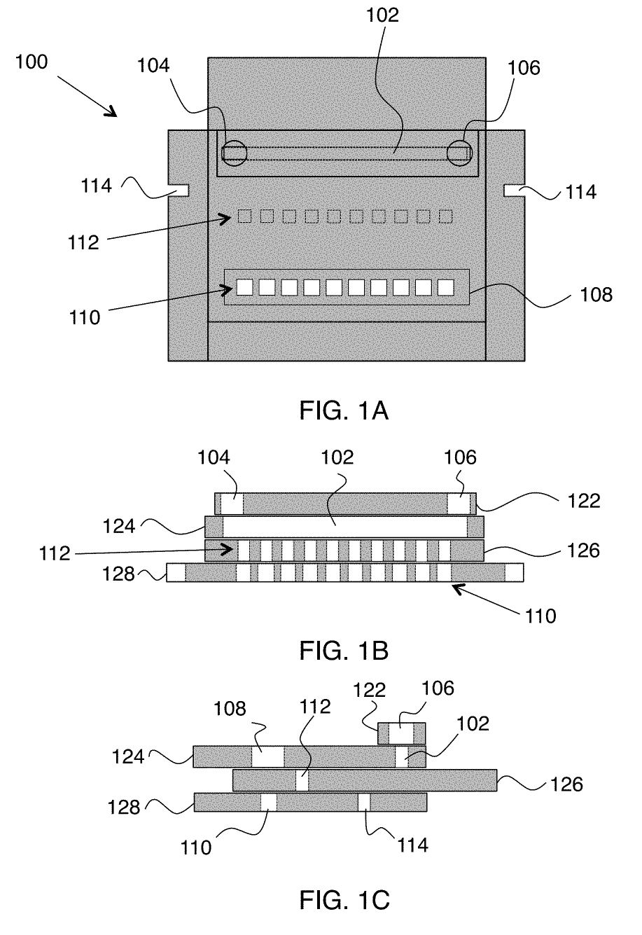

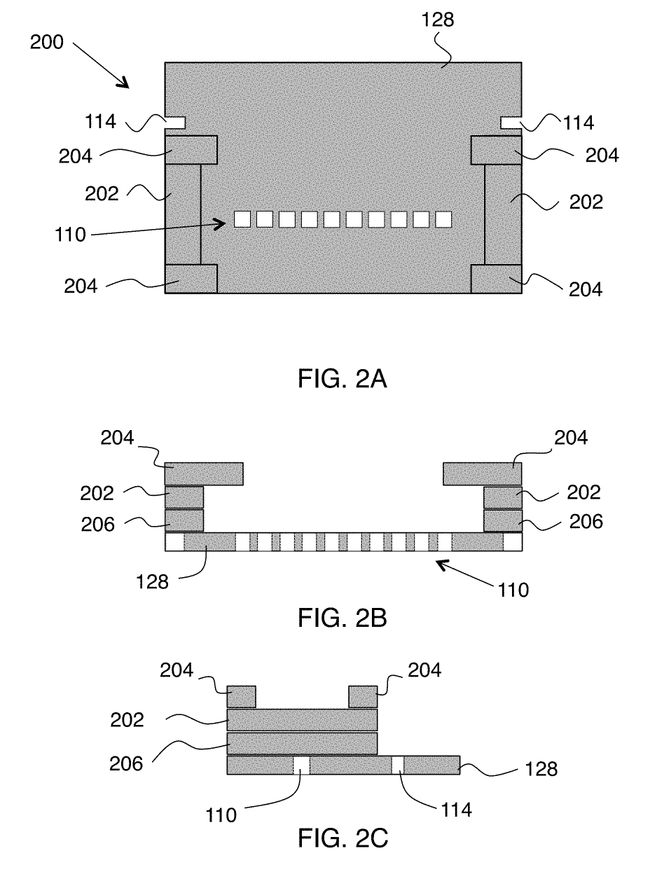

FIGS. 1A-C show a drafting schematic of manifold 100. Here FIG. 1A is a schematic top view, FIG. 1B is a schematic front view, and FIG. 1C is a schematic right view. Its purpose is to accept a sample from a user and deliver precise volumes to each individual dipstick pad. FIGS. 2A-C show a drafting schematic of chassis 200. Here FIG. 2A is a schematic top view, FIG. 2B is a schematic front view, and FIG. 2C is a schematic right view. Its purpose is to secure all the pieces of the manifold and provide alignment with the acrylic housing. These figures are not drawn to scale: the layers are drawn thicker for ease of viewing.

Here the above-mentioned 4-layer structure for the manifold is formed by layers 122, 124, 128, and 128. In the manifold of FIGS. 1A-C, only layers 122 and 124 are permanently bonded together. The chassis has a 4-layer structure formed by layers 202, 204, 206 and 128 and all of these layers in the chassis are permanently bonded together.

Layer 122 of manifold 100 includes sample inlet 104 and sample outlet 106. Layer 124 of manifold 100 includes microfluidic channel 102 and viewing window 108. Layer 126 of manifold 100 includes sample transfer volumes 112. Layer 128 of manifold 100 includes holes 110 for the dipstick pads and alignment cutouts 114 for the opaque housing described below.

Layers 202 and 206 of chassis 200 are configured to laterally align the sliding structure of manifold 100 with respect to holes 110. Layer 204 of chassis 200 is configured to hold the sliding structure of manifold 100 in position vertically during operation of the device, as described in greater detail below.

Layers 122, 124, and 126 of the manifold and layers 202, 204 and 206 of the chassis were made of clear acrylic that was laser-cut to the desired shape, while layer 128 was fabricated out of black opaque acrylic. The choice of opaque acrylic for the bottom layer was intended to eliminate stray light coming from below the device. Note that layer 128 is common to both the manifold and chassis. During operation, the dipstick is placed beneath layer 128 and the pads are inserted into the cutout square holes 110 in layer 128. The size and number of these holes were chosen to match the analyte pads of the specific dipstick brand utilized.

A working schematic of the manifold is shown in FIGS. 3A-C and 4A-D. FIGS. 3A-C provide a front view of the loading process, and FIGS. 4A-D provides a right view of the slipping process. The overall process is as follows: i) the dipstick is placed beneath layer 128 and the pads are inserted into the cutout square holes 110; ii) the user loads the urine sample 302 into the inlet hole of layer 122 (e.g., using a plastic eye dropper). Note that there is no need to measure the volume accurately, as distribution of the required volume is managed by the device; iii) the sample 302 proceeds through the microfluidic channel 102 in layer 124 while simultaneously filling the wells 112 in layer 126 as shown in the sequence from FIG. 3A to FIG. 3C (excess urine exits through the outlet hole 106); iv) after all wells 112 in layer 126 have been filled, layer 126 is slipped across layer 128 as shown in the sequence from FIG. 4A to FIG. 4D; v) at the completion of the slipping process, the volumes held in layer 126 are transferred to the individual dipstick pads held in place by layer 128. The resulting color change in the dipstick pads is schematically shown by the change from gray on FIG. 4C to black on FIG. 4D. The volume transferred by the device is dictated by the dimensions of wells 112 in layer 126. Therefore, should a different volume be required (e.g., for a different brand of dipstick), the volume transferred could be easily adjusted by altering the dimensions of wells 112 in layer 126. The use of square wells allowed the fluid to contact the entire pad and results in a more homogeneous color change. These figures are not drawn to scale: the layers are drawn thicker for ease of viewing.

We bonded layers 122 and 124 using an acrylic sealer to ensure a water-tight seal. To minimize sample loss during the slipping process, we coated the bottom of layer 124 and the top of layer 128 with Neverwet (Rust-Oleum), a hydrophobic coating. The Neverwet ensures that the water tension is not broken while slipping layer 126 and that the full volume in the wells is transferred. Each acrylic layer was 1.59-mm thick, and layer 128 was 160 mm×50 mm. To avoid imaging through potentially cloudy and/or scratched acrylic caused by repeated usage and to ensure complete release of the water tension, a rectangular viewing window 108 was cut out of layer 124 above the dipstick holes 110 (visible in the top view of FIG. 1A. To make the device reusable (i.e., capable of testing multiple dipsticks), after running a urinalysis test, the layers of the manifold/chassis were wiped with a 10% bleach solution and then de-ionized water. This process sanitized the slides and prevented sample contamination. The opaque housing (discussed in the next section) never comes into contact with the urine sample and therefore does not need to be sanitized in between runs.

### B2.3b) Acrylic Housing for Lighting Control

We housed the portion of the manifold that holds the dipstick inside a black opaque box. The box eliminates variations in the lighting conditions, thus eliminating the need to capture a calibration chart with each image or use a complex algorithm that is robust to various lighting conditions; only a single calibration event corresponding to the specific dipstick brand being imaged is needed for the entire lifetime of the device. The placement of the acrylic housing 502 over the manifold is shown in FIG. 5. Notably, microfluidic channel 102 extends beyond housing 502 for easy sample loading. Each side of housing 502 was individually lasercut and then assembled via interlocking cutouts, as shown in FIG. 6. Here 504 and 506 are side pieces for housing 502, 510 is the top piece for housing 502 and includes a hole 516 for camera 504, 508 is the back piece for housing 502 and 512 is the front piece for housing 502. Front piece 512 includes a gap 514 to accommodate layers 124, 126 and 128 of the manifold as shown on FIG. 5. Front piece 512 can engage with cutouts 114 on manifold 100 to provide alignment. Interlocking cutouts allow the box to be easily disassembled and reassembled; therefore, the box can be packaged in individual pieces, which allows for easier storage and/or shipping.

### B2.3c) Geometric Cut-Outs for Timing Control

To enable precise timing of when the urine contacts the dipstick pads after slipping, we affixed portions of stickers of well-defined geometric shape to the tops of both layers 124 and 126 as shown in FIGS. 7A-D. Incomplete half-circle stickers 704a and 704b were placed at the end of layer 124; their complements 702a and 702b were placed at the end of layer 126. Completed half-circles 706a and 706b are formed at the completion of the slipping process (i.e., when the sample volumes are deposited on the dipstick pads), as shown in the sequence from FIG. 7A to FIG. 7D. The analysis software is programmed to identify images of complete half-circles (i.e., completed timing stickers), and uses this cue to start the timer for reading the results.

### B2.3d) End User Operation

When running a urinalysis dipstick test, the end user performs the following steps: 1) Place the dipstick underneath layer 128 and push the pads through the holes. 2) Place the acrylic housing 502 over the manifold/chassis. 3) Place the mobile computing device (or other camera) 504 over the hole 514 on top of the box and turn on video recording. 4) Fill the microfluidic channel with the urine sample using an eye dropper. 5) Slide layer 126 (layer 126 will become flush with the acrylic box 502 when sliding is complete so the user doesn't have to gauge how far to slide). 6) After waiting two minutes (or more), transfer the resulting video to a personal computer. 7) Input the video into the software. While the end user is still responsible for a number of steps, they are all performed either prior to the urine being deposited onto the dipstick or after the test is complete, which eliminates the user error typically associated with dipstick results. Note that steps 6 and 7 would be eliminated if the software were integrated into a mobile application as opposed to an offline system. With a mobile application, the user would simply need to press a ‘start’ button during step 3 and the software would automatically display the results on the screen at the conclusion of the test.

### B2.4) Software Design

We developed software to analyze videos of urinalysis dipstick tests performed with the complete urinalysis device (manifold, chassis, and acrylic housing). The software accomplishes two main tasks: 1) it keeps track of the time when the sample is deposited on the dipstick and the appropriate readout times of all analytes; 2) it analyzes the colors of the dipstick pads and compares them to the reference chart provided by the manufacturer. Since these tasks are done automatically, the user error associated with color differentiation and accurate timing is eliminated.

The overall process of the software is as follows. First, the software determines the time at which the urine sample is deposited onto the dipstick pads (i.e., the starting time) by identifying the first frame where a completed timing sticker exists. Next, for each analyte, the software ascertains the frame corresponding to the readout time provided by the manufacturer. The software then locates the corresponding dipstick pad. For each dipstick pad, comparisons are made between the color of that pad and the color of each corresponding candidate solution on the reference chart. Each color is represented as a three-element vector in the RGB color space. We find these vectors by averaging several pixels of the pad or candidate solution. The angle between vectors is used as a metric of similarity; the smaller the angle, the more similar the colors. This metric is robust to changes in the intensity of illumination, as it is independent of the vector magnitudes. We report the index of the candidate solution with the smallest angle (i.e., whose color is most similar to the candidate pad). Practice of the invention does not depend critically on the color recognition method employed, and any other color recognition method can also be used as an alternative to the method of this example.

The algorithm implemented by the software includes two parts: initialization and analysis. The initialization is performed only once to characterize the device/dipstick pair; whereas, the analysis is performed for each dipstick assay. The initialization results are utilized by the analysis steps, which are performed on each dipstick assay. The analysis part of the algorithm is completely automated: it accepts a video clip as an input and reports the results by displaying the abbreviation for each dipstick pad (e.g., LEU, GLU) along with the index and actual value of the solution (e.g., 1 (100 mg/dL), 2 (250 mg/dL)) for the corresponding pad. The steps of the algorithm are summarized in Algorithm 1. A detailed description of each step follows.

Algorithm 1: Steps for analyzing urinalysis dipstick video

```
Initialization
      Step 1: Create template image
      Step 2: Measure relative offsets between dipstick pads
      and timing sticker
            (a) straighten image
            (b) determine offsets
      Step 3: Capture and store reference chart data
Analysis
      Step 1: Correct aspect ratio of video
      if aspect ratio <= 1
            then rotate image by 90°
      end
      Step 2: Identify start time and correct video
      orientation
      for each frame
            Calculate normalized cross-correlation (NCC)
            if maximum of NCC >= threshold (0.85)
                  start timer
                  correct orientation
                  continue
            end
      end
      Step 3: Record and analyze the results
      for each dipstick pad
              (a) identify frame for readout time
              (b) straighten image
              (c) locate dipstick pad and record color
              (d) compare measured color to reference chart
      end
```

### B2.4a) Initialization

### Step 1: Create Template Image

To analyze whether a completed timing sticker is present in a given frame, we utilized the normalized cross-correlation function. Normalized cross-correlation (NCC) is a standard approach to searching for a known signal in a set of data. It accepts as its input two images: a template image (the known signal) and a search image (the set of data). It returns an image of the same size as the search image where the value of each pixel quantifies how well the template image matched the local region in the search image. The output values range between 1 (a perfect match) and −1 (a perfect anti-match). For this application, the template image is an image of a completed timing sticker, and the search images are frames from the urinalysis video. We created the template image by cropping two completed timing stickers from an image of the manifold and averaging them. Notably, in an image with completed timing stickers, the location of the maximum of the NCC in the upper half of the image corresponds to the location of the top timing sticker. Similarly, the location of the maximum of the NCC in the lower half of the image corresponds to the location of the bottom timing sticker.

### Step 2: Measure Relative Offsets Between Dipstick Pads and Timing Sticker

(a) We chose a frame with completed timing stickers to determine the position of the dipstick pads relative to the timing stickers. The image was initially straightened to correct for small rotations between the camera and the dipstick caused by inconsistencies in their alignment. To straighten the image, we found the locations of the maximum of the NCC in the upper and lower halves of the frame: points (x₁, y₁) and (x₂, y₂), respectively. That is, we located both timing stickers. The frame was then rotated about (x₂, y₂) so that (x₁, y₁) was positioned directly above (x₂, y₂).

(b) The pixel offset between the timing sticker in the lower half of the image, located at (x₂, y₂), and the center of each dipstick pad was measured and recorded. A special case was made for the glucose (GLU) pad, whose hydrophobicity often leads to nonuniform wetting of the pad. This apparent hydrophobicity is evidenced by the urine consistently forming a bead on the pad. This phenomenon results in the sample remaining on the edge of the pad closest to the microfluidic channel when liquid is transferred to the GLU pad during sample delivery. To account for this abnormal distribution, the detection point for the GLU pad was moved closer to the edge of the pad and inward.

### Step 3: Capture and Store Reference Chart Data

We captured an image of the manufacturer's reference chart with the mobile computing device in bright, white-light conditions. From this image, we calculated and stored the RGB vectors for all candidates. The proper readout times, as listed on the reference chart, were manually recorded and stored.

### B2.4b) Analysis

### Step 1: Correct Aspect Ratio of the Video

The mobile computing device can capture videos in four possible orientations. For consistency within all algorithm steps, all images (i.e., frames from the video) were required to be of the same orientation. Therefore, if the aspect ratio of the images was less than one, all frames of the video were rotated by 90°. This step forces all the video frames into one of two orientations, denoted (a) or (b).

### Step 2: Identify Start Time and Correct Video Orientation

The start time (i.e., when the sample volumes are deposited onto the dipstick pads) was assumed to be the first frame that contained completed timing stickers. For each frame, we calculated the maximum of the NCC between the frame and the template image. Additionally, we calculated the maximum of the NCC between the frame and the template image after rotation by 180°. The timestamp for the first frame where the maximum of the NCC exceeds a threshold of 0.85 was considered the starting time. The orientation was confirmed based on whether the maximum of the NCC that exceeded the threshold corresponded to that of the original template image (video orientation a), or its 180°-rotated counterpart (video orientation b).

### Step 3: Record and Analyze the Results

### For Each Dipstick Pad (Analyte):

(a) Using the frame rate of the camera, the predetermined readout times, and the starting time determined in step 2, we identified the frame that corresponded to the correct readout time.
(b) We straightened the image to correct for small rotations, as was done in initialization step 2.
(c) Using the pixel offsets determined in initialization step 2, we identified the location of the dipstick pad. A 3×3 grid centered at this point was averaged to compute the RGB vector for this analyte.
(d) We used the previously described color-matching technique to compare the analyte RGB vector to all candidate RGB vectors from the reference chart. The index of the best match was reported.

### B3) Results and Discussion

### B3.1) Demonstration of Volume Control

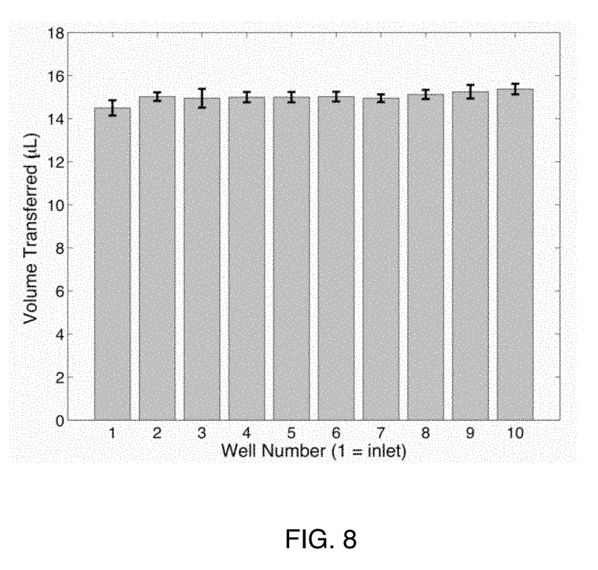

To validate the consistency of the volume transferred by the manifold, we loaded a urine sample, slipped layer 126, and measured the volume transferred by each well 112 using a calibrated pipette. The wells 112 in layer 126 were laser-cut with dimensions of 3.07 mm×3.07 mm×1.59 mm to transfer the optimal volume of 15 μL. The mean volumes transferred by each well (over seven trials) are presented in FIG. 8. Here well 1 is closest to the inlet 104 while well 10 is closest to the outlet 106. The mean volume transferred by each well was within 0.5 μL of the designed value of 15 μL, and all standard deviations were below 0.5 μL. Well 1 differs the most from the designed value; we believe this discrepancy could be caused by a slight retraction of the sample as the eyedropper is removed after loading.

B3.2) Demonstration of timing control

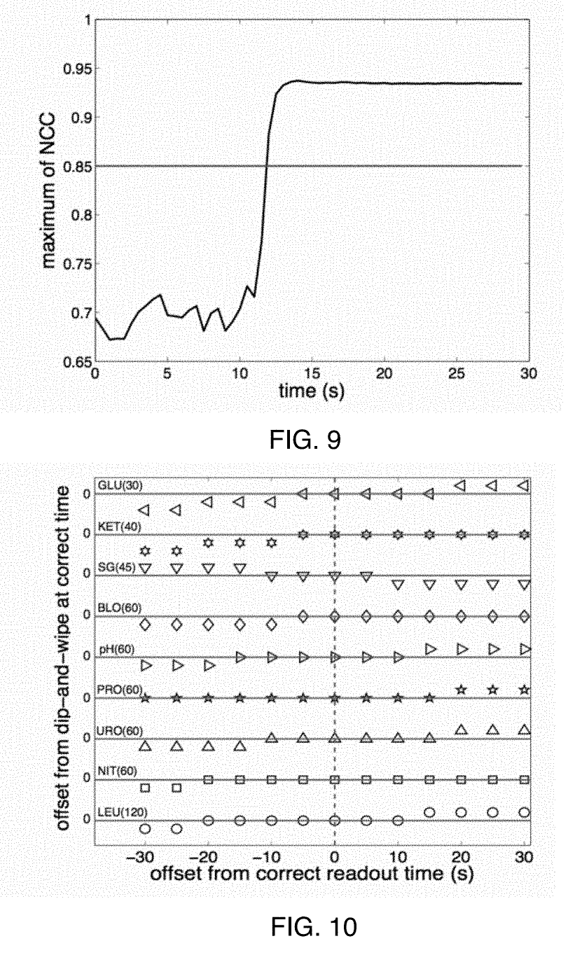

The readout timing for each dipstick pad was based on the manufacturer's directions (i.e., the readout times provided by the manufacturer). We validated the operation of these timing markers in two ways. First, we confirmed visually that the maximum of the NCC crosses a threshold of 0.85 at the same moment that the urine sample is deposited onto the dipstick pads; a representative progression of the maximum of the NCC over time is presented in FIG. 9, where the horizontal line indicates the threshold value.

Secondly, we tracked the results of each dipstick pad, inside the full device, as a function of time using level 2 standard urine (FIG. 10). Here median results over 15 trials are shown. Time is represented as an offset from the readout time provided by the manufacturer and was measured using the timing mechanism and software. All results were compared to the standard dip-and-wipe results read at the recommended time, which are indicated by horizontal lines for each analyte. Notably, all pads match the dip-and-wipe result at a time offset of zero (indicated by vertical dashed line), where they cross the corresponding horizontal line. These results indicate that the timing stickers produce accurate readout times. These data also demonstrate the effects of incorrect timing for each analyte: that is, reading the results at the incorrect time often leads to erroneous results. Recall that the BIL pad does not well match the reference chart, resulting in consistently problematic results and, therefore, the results of the pad were excluded.

TABLE 2
Results of urinalysis performed with the complete
device and analyzed with the developed software.

| | dip/wipe L1 | this work L1 | dip/wipe L2 | this work L2 |
|---|---|---|---|---|
| LEU | 2 ± 0.46 | 2 ± 0.35 | 4 ± 0 | 4 ± 0.49 |
| NIT | 1 ± 0 | 1 ± 0 | 2 ± 0 | 2 ± 0 |
| URO | 1 ± 0 | 1 ± 0 | 3 ± 0 | 3 ± 0 |
| PRO | 1 ± 0 | 1 ± 0.41 | 3 ± 0 | 3 ± 0.52 |
| pH | 2 ± 0.49 | 2 ± 0 | 5 ± 0.49 | 5 ± 0.49 |
| BLO | 1 ± 0.35 | 1 ± 0.5 | 5 ± 0.46 | 5 ± 0.49 |
| SG | 5 ± 0.41 | 6* ± 0.59 | 3 ± 0 | 3 ± 0.56 |
| KET | 1 ± 0 | 1 ± 0 | 5 ± 0.64 | 5 ± 0.65 |
| GLU | 1 ± 0 | 1 ± 0 | 3 ± 0 | 3 ± 0.56 |
| differences | N/A | 1 | N/A | 0 |
| vs. | | | | |
| dip/wipe | | | | |

Results are presented for two standard levels of urine (L1: level 1, L2: level 2). The median values and standard deviations over 15 trials are reported.
A * indicates values that differ from the dip-and-wipe method. The total number of analyte results that differ from the dip-and-wipe method are reported in the bottom row.
LEU: leukocytes, NIT: nitrite, URO: urobilinogen, PRO: protein, BLO: blood, SG: specific gravity, KET: ketones, GLU: glucose.

### B3.3) Demonstration of Full Device

Finally, we tested the utility of the full urinalysis device (including the developed software) with both level 1 and level 2 standard urines; the results are presented in Table 2 (median values over 15 trials). Results of a dip-and-wipe test are reported for comparison. There is only a single error in the L1 results (for specific gravity) and no errors in the L2 results. We calculated p-values for each analyte using a two-sided Fisher's exact test to evaluate the similarity between the dip-and-wipe results and the results obtained using our urinalysis device. The p-values for all analytes were above 0.7, which shows that there is not a statistically significant difference between the two sets of data. The error in the reported specific gravity for L1 most likely results from the similar coloring of candidate solutions 5, 6, and 7 in the reference chart associated with the amount of analyte added by the manufacturer (the exact amount was not specified). The angles between candidate solutions 5, 6, and 7 are 1.4° and 1.3°, respectively, which suggests that their separation in color space is too small to distinguish reliably. As a point of comparison, the angles between candidate solutions 1, 2, 3, 4, and 5 are 10.9°, 12.9°, 5.9°, and 4.9°, respectively. Overall, the manifold, coupled with the software, produces results that are accurate and consistent, similar to a properly performed dip-and-wipe method.

The results presented in Table 2 were all obtained using a single device (a single box and manifold/chassis). In between sample runs the slides were sanitized with a 10% bleach solution as described previously. While the manifold and chassis are reusable, their lifetime is limited by the hydrophobic coating: friction caused by slipping the layers eventually causes the coating to delaminate. We were able to run approximately 15 tests before noticeable delaminating occurred (typically at the edges of the manifold), and we were able to run approximately 30 tests before the delaminating led to leakage. It should be noted that when the coating delaminates, the slides are not damaged; the remaining coating can be removed and a new coating can be applied.

### B4) Conclusion

The all-acrylic, micro-volume urinalysis device described herein controls the many aspects of dipstick urinalysis that are prone to user error (volume control, timing control, lighting control, and color differentiation), thus making it suitable for a home environment or a low-resource area. With the user error removed, the results are more reliable and could potentially encourage the medical community to accept dipstick results reported by patients. Because the device is compatible with off-the-shelf dipsticks, it could be adopted by the medical community quickly. The device only requires a one-time calibration event, making the process even simpler for the patient. Currently the entire algorithm is performed on a personal computer and is therefore not yet fully amenable to low-resource settings.

The current device has been optimized for a specific brand of dipstick, and the holder was made to fit a specific brand of mobile phone. Different dipsticks may have a different number of pads and may have different spacing between pads. Therefore, the number and spacing of holes in layer 128 would need to be slightly modified for each brand. Additionally, the pads of different dipstick brands can vary; hence, the optimal volume for accuracy may deviate from the value used here and would need to be experimentally determined. The dimensions of holes 112 in layer 126 would need to be altered to transfer this optimal volume. Altering the design of holes in layers 126 and 128 would be simple given the fast and low-cost nature of laser-cutting acrylic. Finally, the mobile phone holder can be easily adapted to fit a range of phone options. Given the simple interlocking form of the box, only the top would need to be redesigned as this part merely aligns the camera with the dipstick.

1. Apparatus for analysis of a liquid sample, the apparatus comprising:
a sample holder configured to accept an analysis dipstick having two or more colorimetric test patches;
wherein the sample holder includes a slidable mechanism having a first configuration wherein a liquid sample can be provided to a sample chamber in the sample holder and having a second configuration wherein a predetermined volume of the liquid sample is individually delivered from the sample chamber to each of the colorimetric test patches of the analysis dipstick;
an opaque housing disposed to prevent ambient light from reaching the analysis dipstick when one or more color images of the analysis dipstick are acquired.

2. The apparatus of claim 1, further comprising a camera configured to provide the one or more color images of the analysis dipstick.

3. The apparatus of claim 2, wherein the camera is included on a mobile electronic device.

4. The apparatus of claim 2, wherein the camera is integrated with the opaque housing.

5. The apparatus of claim 2, wherein the camera is configured to further provide a magnified optical image of the liquid sample.

6. The apparatus of claim 5, further comprising a processor configured to:
provide automatic recognition of features of the magnified optical image; and to
provide results from the automatic recognition of features to a user.

7. The apparatus of claim 1, wherein the liquid sample is selected from the group consisting of: human urine sample, animal urine sample, human blood sample, animal blood sample and water sample.

8. The apparatus of claim 1, further comprising a processor configured to:
provide a comparison of the one or more color images of the analysis dipstick to one or more reference images and to
provide results from the comparison to a user.

9. The apparatus of claim 8, wherein the processor is further configured to perform a color calibration.

10. The apparatus of claim 9, wherein the color calibration is a one-time calibration.

11. The apparatus of claim 1, wherein the sample holder further includes one or more optically recognizable markers having:
a first shape when the sample holder is in its first configuration; and having
a second shape when the sample holder is in its second configuration.

12. The apparatus of claim 11, further comprising a processor configured to:
acquire the one or more color images of the analysis dipstick at one or more predetermined times after the one or more optically recognizable markers earliest have their second shape.

13. The apparatus of claim 11, wherein the second shape of the optically recognizable markers is asymmetric, whereby determining orientation of the color images is facilitated.

14. The apparatus of claim 1, wherein the opaque housing is configured as a plurality of planar members comprising plastic and having interlocking cutouts on their edges for assembly to form the housing.

15. The apparatus of claim 1, wherein the sample holder is configured as a stack of two or more planar members comprising plastic, and wherein the first and second configurations are defined by sliding one of the planar members relative to the other planar members.

16. The apparatus of claim 1, wherein the analysis dipstick is a commercially available analysis dipstick, and wherein the sample holder is configured to mechanically mate with the commercially available analysis dipstick.
